(19) United States
(12) Patent Application Publication
(10) Pub. No.: US 2023/0045719 A1
(43) Pub. Date: Feb. 9, 2023

(54) SYSTEMS AND METHODS FOR PRE-FILLED MEDICAL DELIVERY DEVICES

(71) Applicant: Koska Family Limited, East Sussex (GB)

(72) Inventors: Marc Andrew Koska, Dartmouth (GB); Jay S. Walker, Ridgefield, CT (US); Jeff Price, Windermere, FL (US)

(21) Appl. No.: 17/960,111

(22) Filed: Oct. 4, 2022

Related U.S. Application Data
(63) Continuation of application No. PCT/US2021/025683, filed on Apr. 3, 2021.
(60) Provisional application No. 63/028,506, filed on May 21, 2020.
(60) Provisional application No. 63/005,434, filed on Apr. 5, 2020.

(51) Int. Cl. A61M 5/28; A61M 5/34

(52) U.S. Cl. CPC A61M 5/282; A61M 5/34; A61M 5/288; A61J 1/067

(57) ABSTRACT

A pre-filled medical delivery assembly assembled and configured to allow delivery of a single dose of a therapeutic agent (e.g., vaccine, drug, medicament, etc.) from a Blow-Fill-Seal (BFS) vial to a patient. The delivery assembly generally includes a modular design consisting of separately constructed components cooperatively arranged and coupled to one another.

### CROSS-REFERENCE TO RELATED APPLICATIONS

This application claims benefit and priority under 35 U.S.C. § 120 to, and is a Continuation of, International Patent Application No. PCT/US2021/025683 filed on Apr. 3, 2021 and titled “SYSTEMS AND METHODS FOR PRE-FILLED MEDICAL DELIVERY DEVICES”, which itself claims priority under 35 U.S.C. § 119(e) to, and is a Non-provisional of (i) U.S. Provisional Patent Application No. 63/005,434 filed on Apr. 5, 2020 and titled “SYSTEMS AND METHODS FOR PRE-FILLED MEDICAL DELIVERY DEVICES” and (ii) U.S. Provisional Patent Application No. 63/028,506 filed on May 21, 2020 and titled “SYSTEMS AND METHODS FOR PRE-FILLED MEDICAL DELIVERY DEVICES”, each of which is hereby incorporated by reference herein in its entirety.

### BACKGROUND

Every year, millions of people become infected and die from a variety of diseases, some of which are vaccine-preventable. Although vaccination has led to a dramatic decline in the number of cases of several infectious diseases, some of these diseases remain quite common. In many instances, large populations of the world, particularly in developing countries, suffer from the spread of vaccine-preventable diseases due to ineffective immunization programs, either because of poor implementation, lack of affordable vaccines, or inadequate devices for administering vaccines, or combinations thereof.

Some implementations of immunization programs generally include administration of vaccines via a typical reusable syringe. However, in many situations, particularly in developing countries, the administration of vaccines occur outside of a hospital and may be provided by a non-professional, such that injections are given to patients without carefully controlling access to syringes. The use of reusable syringes under those circumstances increases the risk of infection and spread of blood-borne diseases, particularly when syringes, which have been previously used and are no longer sterile, are used to administer subsequent injections. For example, the World Health Organization (WHO) estimates that blood-borne diseases, such as Hepatitis and human immunodeficiency virus (HIV), are being transmitted due to reuse of such syringes, resulting the death of more than one million people each year.

Previous attempts at providing single-use or disposable injection devices to remedy such problems in the industry have achieved measurable success but have failed to adequately remedy the existing problems. Pre-filled, single-use injection devices manufactured via injection molding or Form-Fill-Seal (FFS) processes, such as the Uniject™ device available from the Becton, Dickinson and Company of Franklin Lakes, N.J., for example, while offering precise manufacturing tolerances in the range of two thousandths of an inch (0.002-in; 50.8 μm) to four thousandths of an inch (0.004-in; 101.6 μm)—for hole diameters in molded parts, require separate sterilization processes (e.g., gamma radiation) that are not compatible with certain fluids, provide production rates limited to approximately nine thousand (9,000) non-sterile units per hour, and can be provided to an end-user for approximately one dollar and forty cents ($1.40) per dose/unit.

### BRIEF DESCRIPTION OF THE DRAWINGS

An understanding of embodiments described herein and many of the attendant advantages thereof may be readily obtained by reference to the following detailed description when considered with the accompanying drawings, wherein:

FIG. 1A, FIG. 1B, FIG. 1C, and FIG. 1D are various views of a pre-filled medical delivery assembly according to some embodiments;

FIG. 2A, FIG. 2B, FIG. 2C, and FIG. 2D are various views of a pre-filled medical delivery device according to some embodiments;

FIG. 3A and FIG. 3B are side and perspective cross-sectional views of a portion of a BFS vial according to some embodiments; and

FIG. 4 side cross-sectional view of a portion of an administration assembly according to some embodiments.

### DETAILED DESCRIPTION

### I. Introduction

Embodiments of the present invention provide systems and methods for pre-filled medical delivery devices and/or assemblies that overcome drawbacks of current delivery devices and methods. For example, the pre-filled medical delivery assemblies of some embodiments may include a Blow-Fill-Seal (BFS) vial or bottle coupled to a specialized collar that facilitates coupling of an administration member (e.g., a needle) to the BFS vial. In some embodiments, such a pre-filled medical delivery assembly may be selectively actuated by engagement of an administration member, causing the administration member to axially advance and pierce the BFS vial. Utilization of such systems that employ BFS vials may be advantageous and may address various shortcomings of previous systems.

BFS vials may, for example, offer a less expensive alternative to typical vials or devices created via other manufacturing techniques. In some embodiments, BFS vials (e.g., due to the nature of the BFS manufacturing process) may not require separate sterilization (e.g., and may accordingly be compatible with a wider array of fluids), may provide enhanced production rates of sterile/aseptic units per hour, and/or may be provided to an end-user for significantly lower per dose/unit costs. In some embodiments, these advantages may come with attendant drawbacks of reduced manufacturing tolerances and other disadvantages of utilizing a “soft” plastic (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)—for linear dimensions, e.g., in accordance with the standard ISO 2768-1 “General tolerances for linear and angular dimensions without individual tolerance indications” published by the International Organization for Standardization (ISO) of Geneva, Switzerland (Nov. 15, 1989) and/or may not be readily adaptable to form certain mating features such as standardized threads. In some embodiments, these drawbacks and/or the deficiencies of prior systems may be advantageously addressed by specific features, configurations, and/or components as described hereinafter.

### II. Pre-Filled Medical Delivery Assemblies

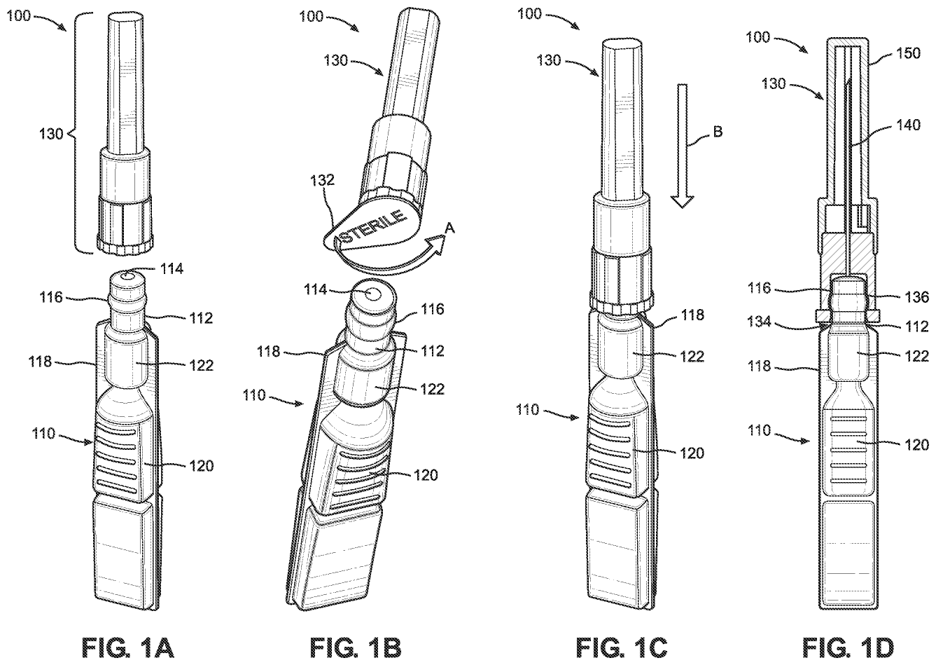

Referring initially to FIG. 1A, FIG. 1B, FIG. 1C, and FIG. 1D, various views of a pre-filled medical delivery assembly 100 according to some embodiments are shown. In some embodiments, the pre-filled medical delivery assembly 100 may comprise various inter-connected and/or modular components such as a BFS component, bottle, and/or vial 110 comprising and/or defining a vial neck 112, a fluid seal 114, a mounting flange 116, a bottle flange 118, a collapsible reservoir 120, and/or a dispensing reservoir 122. According to some embodiments, the pre-filled medical delivery assembly 100 may comprise an administration module or component 130 that is, e.g., maintained as a closed and/or sterile component via a seal 132 (e.g., a foil, wax, paper, and/or other thin, pierceable, tear-able, and/or removable object or layer coupled to the administration component 130) that seals an interior volume or socket 134 disposed at a first end thereof. According to some embodiments, the socket 134 may comprise and/or define (e.g., on or in an interior surface thereof) a shaped seat 136 that is configured to accept the mounting flange 116 of the BFS vial 110 (e.g., in the case that the neck 112 of the BFS vial 110 is inserted into the socket 134). In some embodiments, the administration component 130 may comprise and or house a canula or needle 140 selectively shrouded by a cap 150.

In some embodiments, the collapsible reservoir 120 may be filled (fully or partially) with a fluid or other agent (not separately shown) to be delivered, e.g., to a patient (not shown). According to some embodiments, the fluid may be injected into the BFS vial 110 in a sterile environment during manufacture via a BFS process and sealed within the BFS vial 110 via the fluid seal 114. The fluid seal 114 may comprise a portion of the molded BFS vial 110 for example that is configured to be pierced to expel the fluid, e.g., such as by providing a flat or planar piercing surface and/or by being oriented normal to an axis of the BFS vial 110 (and/or the pre-filled medical delivery assembly 100). In some embodiments, the fluid seal 114 may comprise a foil, wax, paper, and/or other thin, pierceable object or layer coupled to the BFS vial 110. According to some embodiments, the neck of the BFS vial 110 may comprise the mounting flange 116 such as, e.g., the “doughnut”-shaped exterior flange depicted. The mounting flange 116 may, for example, provide a radially elastic mating surface that is operable to provide a selective engagement or fit within the socket 134 of the administration component 130.

According to some embodiments, the fluid may generally pass between the collapsible reservoir 120 and the connected dispensing reservoir 122. In some embodiments, a juncture, valve, constriction, and/or passage (not separately labeled in FIG. 1A, FIG. 1B, FIG. 1C, or FIG. 1D) between the dispensing reservoir 122 and the collapsible reservoir 120 may restrict flow such that the fluid may readily enter one of the dispensing reservoirs 122 and the collapsible reservoir 120 but may not readily return to the other reservoir 120, 122. Such a constriction may in some embodiments, provided advantages as described herein. In some embodiments, the constriction may not be necessary or desirable, such as in the case that the collapsible reservoir 120 and the dispensing reservoir 122 are formed and/or combined as a single, unconstructed reservoir, e.g., a single fluid reservoir (not shown).

According to some embodiments, the pre-filled medical delivery assembly 100 may include a modular design consisting of separately constructed components 110, 130 cooperatively arranged and coupled to one another. As depicted in FIG. 1A, for example, the BFS vial 110 and the administration component 130 may be manufactured, packaged, shipped, stored, and/or provided as separate components. In such a manner, the administration component 130 may not need to be stored or shipped in accordance with often restrictive requirements imposed on medicaments and may accordingly reduce the amount of space required for such specialized storage and/or shipping. The administration component 130 may also or alternatively be manufactured, stored, and/or shipped in advance (e.g., at a first time) while the BFS vial 110 that is pre-filled with the fluid may be manufactured, stored, and/or shipped at a later time (e.g., a second time). In some embodiments, the delay between the first time and the second time may be lengthy without causing determinantal effects, as the administration component 130 may be stored, in some embodiments, indefinitely. In such a manner, units of the administration component 130 may be provided to be on-hand in advance of the availability and/or arrival of the BFS vial 110, reducing supply chain constraints in the case of proactive administration component 130 procurement.

In some embodiments, the components 110, 130 may be coupled, e.g., in the field and/or in situ, to provide an active pre-filled (e.g., injectable) medical delivery device. As shown in FIG. 1B, for example, the seal 132 may be removed from the administration component 130 (at “A”) and the administration component 130 (and/or the socket 134 thereof) may be aligned with the neck 112 of the BFS vial 110. According to some embodiments, the administration component 130 may be axially engaged to couple with the BFS vial 110 via application of a mating axial force, as shown in FIG. 1C (at “B”). The administration component 130 may be urged onto the neck 112 of the BFS vial 110, for example, such that the cooperatively shaped seat 136 (e.g., an interior groove) accepts the mounting flange 116, thereby selectively and/or removably coupling the BFS vial 110 and the administration component 130. In some embodiments, the mounting flange 116 may be shaped as an axially elongated rounded exterior flange (e.g., the “doughnut” shape as depicted) and/or the shaped seat 136 may comprise a cooperative and/or mirrored axially elongated rounded interior groove or track.

As depicted in FIG. 1D, for example, the neck 112 of the BFS vial 110 may be urged and/or forced into the socket 134 until the mounting flange 116 becomes seated in (and/or coupled to or mated with) the shaped seat 136 (e.g., a seated position). In such a manner, the fluid seal 114 may be advantageously positioned adjacent to the needle 140 and/or may be engaged with the needle 140. In some embodiments, advancement of the neck 112 of the BFS vial 110 into the socket 134 through to the seated position may cause the needle 140 to pierce the fluid seal 114. According to some embodiments, the mounting flange 116 may be configured as the doughnut shape (as depicted) to provide various advantages to the pre-filled medical delivery assembly 100. The axial elongation of the mounting flange 116 may, for example, provide for a smooth, uniform, and/or less forceful mating process that is less likely to deform the soft plastic neck 112 of the BFS vial 110 and/or may provide for a lengthened mating surface that is more likely to prevent leakage of the fluid. In some embodiments, the mounting flange 116 and the cooperatively shaped and sized shaped seat 136 may permit simple, effective, and/or economic attachment of the needle 140 to the BFS vial 110.

According to some embodiments for example, the administration component 130 may be utilized to couple and/or mate the needle 140 with the BFS vial 110 to provide a mechanism via which the needle 140 may be coupled to the soft plastic BFS vial 110 in a reliable and commercially desirable manner. Due to the nature of the BFS plastic and/or process and/or the small form-factor of the BFS vial 110, for example, providing standardized external threads (not shown) directly on the neck 112 would not be a viable option for it would result in an imprecise, unreliable, and/or non-water tight coupling (i.e., the threads would be deformable even if they could be properly manufactured to within the desired tolerances, which itself is not a likely result) between the BFS vial 110 and, e.g., the administration component 130. Applicant has realized, for example, that “soft” plastics required for the BFS process are not susceptible to machining due to heat deformation of machined features during formation attempts as well as deformation due to mechanical stress during utilization. As such, standardized screw-on needle hubs (not shown) are not readily compatible for attachment to BFS vials 110.

In some embodiments, the needle 140 may comprise a needle shaped and/or sized for at least one of subcutaneous, intramuscular, intradermal, and intravenous injection of the fluid agent into the patient. For ease of explanation and description, the figures and the description herein generally refer to the needle 140 as a needle. However, it should be noted that, in other embodiments, the needle 140 may also or alternatively comprise a nozzle (not shown) configured to control administration of the fluid agent to the patient. The nozzle may comprise a spray nozzle, for example, configured to facilitate dispersion of the fluid agent into a spray. Accordingly, a version of the administration component 130 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a version of the administration component 130 fitted with a droplet nozzle may be useful in the administration of a fluid agent by way of droplets, such as administration to the eyes, topical administration, and the like.

As generally understood, the fluid or drug agent may include any type of agent to be injected into a patient (e.g., mammal, either human or non-human) and capable of producing an effect (alone, or in combination with an active ingredient). Accordingly, the agent may include, but is not limited to, a vaccine, a drug, a therapeutic agent, a medicament, a diluent, an active ingredient, and/or the like. According to some embodiment, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components such as the BFS vial 110 and/or the administration component 130.

According to some embodiments, the administration component 130 (and/or the cap 150) may be composed of a medical grade material. In some embodiments, the administration component 130 (and/or the cap 150) may be composed of a thermoplastic polymer or other “hard” plastic (e.g., greater than 80 on the Rockwell “R” scale), including, but not limited to, polybenzimidazole, acrylonitrile butadiene styrene (ABS), polystyrene, polyvinyl chloride, or the like. In some embodiments, the pre-filled medical delivery assembly 100 may be advantageously manufactured (in mass quantities) in separate parts or portions, namely, at least the “soft” plastic BFS vial 110 portion (e.g., a “first” piece) and the “hard” plastic administration component 130 (e.g., the “second” piece), with such different plastic parts/portions being selectively coupled to administer a medication to a patient.

In some embodiments, fewer or more components 110, 112, 114, 116, 118, 120, 122, 130, 132, 134, 136, 140, 150 and/or various configurations of the depicted components 110, 112, 114, 116, 118, 120, 122, 130, 132, 134, 136, 140, 150 may be included in the pre-filled medical delivery assembly 100 without deviating from the scope of embodiments described herein. In some embodiments, the components 110, 112, 114, 116, 118, 120, 122, 130, 132, 134, 136, 140, 150 may be similar in configuration and/or functionality to similarly named and/or numbered components as described herein. According to some embodiments, the pre-filled medical delivery assembly 100 may comprise the mounting flange 116 but not the collapsible reservoir 120. In some embodiments, the pre-filled medical delivery assembly 100 may comprise the mounting flange 116 but not the dispensing reservoir 122.

### III. Pre-Filled BFS Vials

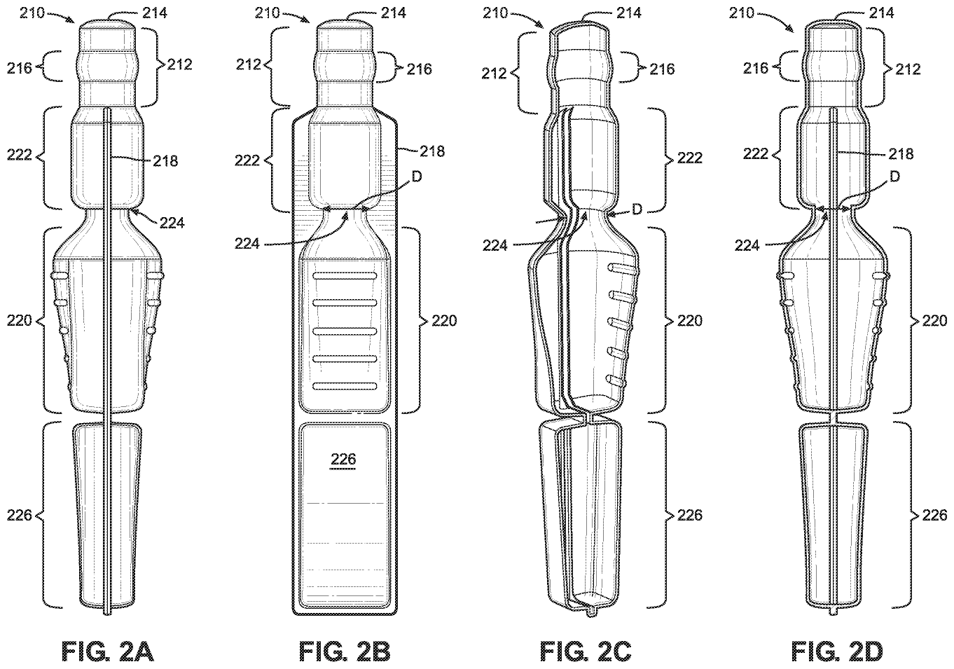

Referring to FIG. 2A, FIG. 2B, FIG. 2C, and FIG. 2D, various views of a pre-filled medical delivery device 210 according to some embodiments are shown. The pre-filled medical delivery device 210 may comprise, for example, a BFS bottle, vial, and/or container manufactured via a BFS process in which a fluid (not separately depicted) is injected into the pre-filled medical delivery device 210 during the manufacturing process (e.g., in a sterile environment). According to some embodiments, the pre-filled medical delivery device 210 may comprise and/or define a neck portion 212 that terminates at a fluid seal 214 disposed at a first end of the pre-filled medical delivery device 210. In some embodiments, the neck portion 212 may comprise and/or define a mounting collar 216 formed as an axially elongated and/or rounded exterior flange or projection, e.g., the example “doughnut” shaped mounting collar 216 as depicted. According to some embodiments, the pre-filled medical delivery device 210 may comprise a bottle flange 218 which may, for example, comprise unmolded portions of fused parison. In some embodiments, the pre-filled medical delivery device 210 may comprise and/or define a first chamber 220 and/or a second chamber 222. In some embodiments, the chambers 220, 222 may be joined by a passage or constriction 224. According to some embodiments, the pre-filled medical delivery device 210 may comprise a label tab 226, e.g., formed and/or disposed at a second end of the pre-filled medical delivery device 210.

In some embodiments, various features of the of the pre-filled medical delivery device 210 may provide distinct advantages. In the case of the neck portion 212, for example, a cylindrical shape (as depicted) may provide for enhanced strength and/or ease of insertion for mating with an administration module (not shown; e.g., the administration component 130 of FIG. 1A, FIG. 1B, FIG. 1C, and/or FIG. 1D herein). According to some embodiments, the shape and/or dimensions of the mounting collar 216 may enable the pre-filled medical delivery device 210 to be readily and/or securely coupled to an administration module such as an injection-enabled module. The mounting collar 216 may, for example, provide for a uniform resistance during an axial mating process. In the case that a more pronounced and/or axially shorter projection were utilized, for example, larger point or instantaneous force levels may be experienced and such elevated force levels may compromise the structural integrity of the soft plastic, BFS-produced, pre-filled medical delivery device 210. In some embodiments, the special shape of the mounting collar 216 may reduce the likelihood that axial forces (e.g., applied to mate the pre-filled medical delivery device 210 with an administration and/or injection module) may cause the pre-filled medical delivery device 210 to collapse, e.g., at the constriction 224, between the first chamber 220 and the label tab 226, and/or at the first chamber 220.

According to some embodiments, the axial length of the mounting collar 216 may also or alternatively reduce the likelihood of leakage. In the case that the pre-filled medical delivery device 210 is punctured (e.g., at the fluid seal 214) and coupled with an administration module, for example, the mounting collar 216 may provide an increased surface contact area. In some embodiments, the increased surface contact area along the neck portion 212 (and including the mounting collar 216) may increase the overall friction force that acts against permitting any leaking fluid from escaping the junction between the pre-filled medical delivery device 210 and the administration module. According to some embodiments, the shape of the mounting collar 216 may also or alternatively reduce the likelihood of leakage by providing an axially elongated surface that more easily and/or uniformly elastically engages within a socket and/or seat. The elongated nature of the mounting collar 216 may, for example, weaken the radial elastic resistive forces of the neck portion 212 along the length of the mounting collar 216, thereby permitting a tighter fit (which decreases leakage) while maintaining ease of selective coupling (and/or uncoupling).

In some embodiments, the constriction 224 may provide various advantages. In the case of a multi-chamber pre-filled medical delivery device 210 as depicted, for example, the constriction 224 may be sized (e.g., at a diameter “D”) to be large enough for a filling needle (e.g., a mandrel; not shown) to pass between the chambers 220, 222 but small enough to restrict the flow of fluid between the chambers 220, 222. In the case that the constriction 224 is sized small enough such that the surface tension forces of a retained fluid (e.g., a liquid medicament) prevent the liquid from readily (e.g., without applied force in addition to a resting gravitational force) passing through the constriction 224, for example, the liquid may advantageously (and/or selectively) be maintained in a single one of the chambers 220, 222. According to some embodiments, it may be desirable to fill the liquid into the pre-filled medical delivery device 210 during the BFS process from the first end of the pre-filled medical delivery device 210 (e.g., the end that is eventually formed into and/or sealed by the fluid seal 214) such that the filling needle passes through the constriction 224 and deposits the liquid into the first chamber 220. The constriction 224 may then, for example, hold the liquid in the first chamber 220, e.g., despite changes in orientation, by having the diameter “D” be sized such that the surface tension of the liquid will prevent the liquid from freely flowing through the constriction 224 in the absence of an additionally applied axial force. The constriction 224 may affect the same result in the case that the pre-filled medical delivery device 210 is filled in the opposite orientation, with the second end being upright (not shown). In either case, the liquid may be retained in the first chamber 220 such that, as the pre-filled medical delivery device 210 continues to be molded and/or sealed (e.g., at the fluid seal 214), the hottest parts of the parison/resin are maintained away from (e.g., not in contact with and/or separated from) the liquid. In such a manner, for example, the liquid may be able to be maintained at lower temperatures than if simply filled into a chamber 220, 222 without taking advantage of the separation distances provided by the constriction 224. As certain medicaments have specific temperature tolerance ranges, such a feature may permit liquids to be filled in the pre-filled medical delivery device 210 that would otherwise not be compatible with the heat realized during the BFS manufacturing process.

According to some embodiments, the constriction 224 may provide advantages in other cases where it may be desirable to maintain the fluid (e.g., a liquid) in a single chamber 220, 222. In some embodiments, it may be desirable to separate a filled liquid from air (and/or another gas) in communication with the liquid within the two chambers 220, 222. During inspection processes, for example, it may be advantageous to have the liquid retained in the second chamber 222 while the gas is separated into the first chamber 220. The second chamber 220 may be advantageously shaped and/or configured to be readily collapsible upon receiving a “squeezing” or inward radial force, and/or may be referred to as an air chamber for example, yet the first chamber 220 may be shaped (and/or may comprise features such as grips and/or textured surfaces) in a manner that interferes with or prohibits appropriate inspection (e.g., external optical inspection) of any contents thereof. According to some embodiments, the second chamber 222 may comprise and/or define a simple cylindrical shape that provides smooth and/or uniform external surfaces that may be more conducive to optical (and/or other) inspection processes. In some embodiments, the liquid may be urged into the second chamber 222 by application of an axial force such that the liquid substantially becomes disposed in the second chamber 222 while the first chamber retains any air and/or gas in the first chamber 220. The second chamber 222 may then be inspected, for example, providing desired data regarding the liquid disposed therein. In some embodiments, the liquid may be similarly advanced to and/or maintained in the second chamber 222 in preparation for injection. A user (not shown) may “flick” or otherwise apply axial force (linear and/or centrifugal) to the pre-filled medical delivery device 210, thereby causing any pre-filled liquid to advance to (and/or remain in) the second chamber 222. The constriction 224 may prevent the liquid from retreating back into the first chamber 220, such that the full pre-filled dose of liquid may be readily viewed and/or perceived in the second chamber 222. In such a manner, in the case that the first chamber 220 is squeezed, the fluid/air/gas therein may exert pressure on the liquid in the second chamber 222 and, in the case that the fluid seal 214 has been punctured and/or otherwise compromised, the liquid may accordingly be forced though the neck portion 212 and be expelled from the pre-filled medical delivery device 210 (e.g., injected, in the case that an injection member is coupled thereto). According to some embodiments, the volume of the first and second chambers 220, 222 may be configured to be approximately two and one quarter milliliters (2.25-ml) and/or the ratio of air/gas to liquid may be configured to be between one hundred and two hundred and fifty percent (100%-250%)—i.e., there may be one to two and one-half times the amount of air in the chambers 220, 222 as there is pre-filled liquid. The larger volume of gas/air may, for example, provide an advantageous compressible driver to expel the liquid from the chamber(s) 220, 222.

In some embodiments, fewer or more components 212, 214, 216, 218, 220, 222, 224, 226 and/or various configurations of the depicted components 212, 214, 216, 218, 220, 222, 224, 226 may be included in the pre-filled medical delivery device 210 without deviating from the scope of embodiments described herein. In some embodiments, the components 212, 214, 216, 218, 220, 222, 224, 226 may be similar in configuration and/or functionality to similarly named and/or numbered components as described herein. According to some embodiments, the pre-filled medical delivery device 210 may comprise the mounting flange 216 but not the constriction 224. In some embodiments, the pre-filled medical delivery device 210 may comprise the constriction 224 but not the mounting flange 216.

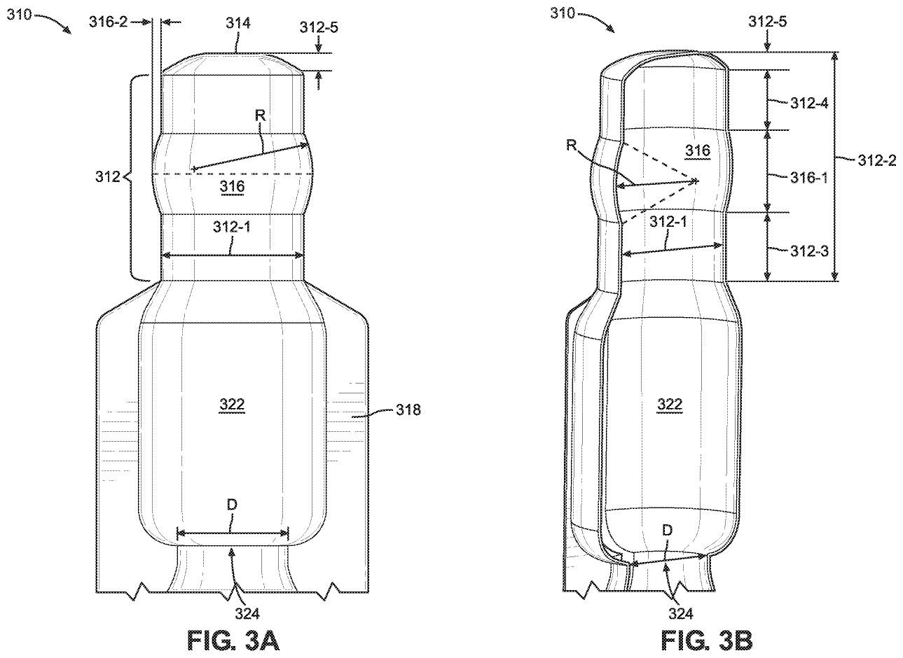

Referring now to FIG. 3A and FIG. 3B, side and perspective cross-sectional views of a portion of a BFS vial 310 according to some embodiments are shown. The portion of the BFS vial 310 may comprise and/or define, for example, a neck 312 having a diameter 312-1, a height or length 312-2, a plurality of intermediate length dimensions 312-3, 312-4, a taper 312-5, and/or an end or seal 314. According to some embodiments, the neck 312 may be substantially cylindrical and/or may be formed or molded as part of the portion of the BFS vial 310. In some embodiments, the neck 312 may comprise and/or define a mounting feature 316. The mounting feature 316 may comprise, for example, a portion of the neck 312 (e.g., defined along a mounting projection length 316-1 thereof) that extends radially outward beyond the diameter 312-1 of the neck 312, e.g., by a protrusion amount 316-2. In some embodiments, the neck 312 may comprise and/or define a portion of an interior volume of the portion of the BFS vial 310, such as may be in communication with a fluid reservoir 322 at a first end thereof. In some embodiments, the fluid reservoir 322 may comprise and/or define a passage 324 (e.g., having a diameter “D”) at a second end thereof.

In some embodiments, such as in the case that the entire BFS vial 310 (not shown in FIG. 3A or FIG. 3B) is between sixty and eight millimeters in length (60-mm to 80-mm), or in some cases approximately seventy millimeters (70-mm) in length, the diameter 312-1 of the neck 312 may be between six and a half millimeters and six and sixty-five hundredths millimeters (6.5-mm to 6.65-mm). According to some embodiments, the diameter 312-1 of the neck 312 may vary, such as having different diameters 312-1 (not shown) below the mounting feature 316 and above the mounting feature 316. In some embodiments, the overall length 312-2 of the neck 312 may be between ten and eleven millimeters (10-mm to 11-mm), or in some cases approximately ten and forty-four hundredths millimeters (10.44-mm). According to some embodiments, the mounting feature 316 may be centered along the length 312-2 of the neck 312 and/or the mounting projection length 316-1 may be between three and four millimeters (3-mm to 4-mm), or in some cases approximately three and forty-five hundredths millimeters (3.45-mm). In some embodiments, a first intermediate length dimension 312-3 may be approximately three and thirty-eight hundredths millimeters (3.38-mm), a second intermediate length dimension 312-4 may be approximately two and fifty-two hundredths millimeters (2.52-mm), and/or the taper 312-5 may be approximately one millimeter (1-mm) in length. According to some embodiments, the protrusion amount 316-2 may be between five and nine tenths millimeters (0.5-mm to 0.9-mm) or in some cases approximately (i) sixty-five hundredths millimeters (0.65-mm) or (ii) eight tenths millimeters (0.8-mm).

According to some embodiments, the protrusion amount 316-2 may be configured to provide a desired amount of resistance upon insertion into a mating element (not shown). In some embodiments, a ratio of the protrusion amount 316-2 to the mounting projection length 316-1 may be configured to provide the desired resistance, elasticity, and/or force (and/or pressure) profile. In a case where the protrusion amount 316-2 comprises eight tenths of a millimeters (0.8-mm), for example, if the mounting projection length 316-1 were sized between one and two times (1× to 2×) the protrusion amount 316-2 (i.e., between eight tenths of a millimeters (0.8-mm) and one and six tenths of a millimeter (1.6-mm)), the profile of the amount of axial force required to push the mounting feature 316 into a seat (not shown) would comprise a sharp buildup of pressure and a sudden release as such a rib-styled mounting feature 316 became seated. In some embodiments, however, the ratio of the mounting projection length 316-1 to the protrusion amount 316-2 may be configured to provide for a more attenuated pressure build up, a lower peak or average pressure, and/or a more gradual release—e.g., to protect the integrity of the soft plastic, BFS-produced, BFS vial 310 and/or to provide for a smooth and repeatable user process. Table 1 shows example pressure values based on an example axial force of two and one quarter pounds (2.25 lbs; 10 N) being required to accomplish a mating of the mounting feature 316 (assuming a single unit of circumferential/lateral length, e.g., for a cylindrical-shaped neck 112; and where the ratio is expressed as a multiplier, for ease of description):

TABLE 1

| Axial | protrusion amount | mounting projection | | |
|---|---|---|---|---|
| Force | 316-2 | length 316-1 | Ratio | Pressure |
| 10 N | 0.8-mm | 0.8-mm | 1× | 12.5 Pa |
| 10 N | 0.8-mm | 1.2-mm | 1.5× | 8.3 Pa |
| 10 N | 0.8-mm | 1.6-mm | 2× | 6.3 Pa |
| 10 N | 0.8-mm | 2.4-mm | 3× | 4.2 Pa |
| 10 N | 0.8-mm | 3.2-mm | 4× | 3.2 Pa |
| 10 N | 0.8-mm | 4.0-mm | 5× | 2.0 Pa |
| 10 N | 0.8-mm | 4.8-mm | 6× | 1.7 Pa |

As shown in Table 1, the pressure exerted on the soft BFS plastic vial 310 can vary significantly, even for a steady or uniform applied force, as the engagement length (i.e., the mounting projection length 316-1; and/or area) varies with respect to the protrusion amount 316-2. According to some embodiments, the ratio of the mounting projection length 316-1 to the protrusion amount 316-2 may be configured to provide for a design pressure corresponding to a desired factor of safety value below an expected failure pressure of the neck 312. The ratio may be advantageously configured, for example, such that the mounting projection length 316-1 is between four and three tenths times (4.3×) and five and three tenths times (5.3×) the protrusion amount 316-2. In some embodiments, the protrusion amount 316-2 may be between sixty-five hundredths to eighty hundredths of a millimeters (0.65-mm to 0.80-mm) and the mounting projection length 316-1 may be between three and four millimeters (3.0-mm to 4.0-mm). According to some embodiments, the required mating force may be configured to be between one and a half pounds (1.5 lbs; 6.7 N) and eight pounds (8 lbs; 35.6 N), by adjustment of the ratio of the mounting projection length 316-1 to the protrusion amount 316-2.

In some embodiments, the distribution of the force and/or pressure over time and/or at points along the mounting projection length 316-1 as it traverses into a seat, e.g., a force and/or pressure profile, may also or alternatively be configured to reduce strain on components of the BFS vial 310. In the case of a smaller the ratio of the mounting projection length 316-1 to the protrusion amount 316-2, such as in the case that the mounting projection length 316-1 is one times (1×) the protrusion amount 316-2 (e.g., the first row in Table 1, above), the entire application of force (and/or pressure buildup) will occur over a small distance (e.g., less than one millimeter (1-mm)). Such a compact distribution of the force/pressure may result in a brief but substantially pressure build-up and a quick (or sudden) release of the pressure upon seating. Particularly for such small form-factor BFS vials 310 and/or for fragile administration components (e.g., small injection needles, etc.), such a compact force/pressure profile may not be desirable. Components may be more likely to incur damage, for example, and/or the user may be more likely to apply over-force, greatly increasing the likelihood of damage.

According to some embodiments, the force/pressure may be configured to be attenuated such as in the case that the mounting projection length 316-1 is configured to approximately five and three tenths times (5.3×) the protrusion amount 316-2 (e.g., the protrusion amount 316-2 is sixty-five hundredths millimeters (0.65-mm) and the mounting projection length 316-1 is three and forty-five hundredths millimeters (3.45-mm)) and the mounting feature 316 is defined as a radially rounded exterior projection having a radius “R”. In some embodiments, the radius “R” may be in the range of four to five millimeters (4-mm to 5-mm) or in some cases approximately four and three tenths millimeters (4.3-mm). Such a radius “R” may, for example, permit the force/pressure to more slowly and/or uniformly build up as the neck 312 advances axially, which may reduce strain on the components and/or may dissuade the user from applying increased force. According to some embodiments, having the radius “R” extend the curvature of the mounting feature 316 from one extent to the other along the mounting projection length 316-1 may provide for not only an attenuated force/pressure build up, but a smooth and uniform release or easing of the force/pressure as the apex of the curvature advances into the corresponding seat. In some embodiments, the mounting projection length 316-1 may be configured to comprise between thirty and forty-five percent (30%-45%) of the side wall length of the neck 312 (i.e., the intermediate length dimensions 312-3, 312-4 plus the mounting projection length 316-1). According to some embodiments, the mounting projection length 316-1 may be configured to comprise approximately thirty-seven percent (37%) of the side wall length of the neck 312.

In some embodiments, the passage 324 (e.g., having the diameter “D”; e.g., an inside diameter) may be sized and/or configured to permit liquid (not shown) disposed in the fluid reservoir 322 to remain in the fluid reservoir 322 when oriented vertically and thus in the case that gravitational force urges the liquid downward toward the passage 324. The diameter “D”, for example, may be sized to be between two and four and one half millimeters (2-mm to 4.5-mm), depending upon the designed density and/or surface tension of the liquid, expected temperature exposure ranges, and/or designed volume/depth of the liquid in the fluid reservoir 322. At an approximate depth of liquid of ten millimeters (10-mm) and assuming a liquid at standard temperature, pressure, and/or having a density comparable to water, for example, the diameter “D” may be configured to be approximately three millimeters (3-mm), which would prevent gravitational forces from breaking the surface tension of the liquid and pulling through the passage 324. In other words, the passage 324 would be substantially small enough that it would retain the liquid in the fluid reservoir 322, e.g., unless and until additional forces were applied (changes in pressure such as due to a squeezing force, application of additional axial forces, e.g., by a user, etc.). In some embodiments, the passage 324 may be configured to both be large enough to permit passage of a BFS process filling needle (e.g., mandrel) and be small enough to retain the liquid in the fluid chamber 322. In the case of a filling needle/mandrel (not shown) having an outside diameter of three millimeters (3-mm), for example, the passage 324 may be sized at approximately four millimeters (4-mm) such that the needle/mandrel may freely pass through the passage 324 but also such that a therapeutic and/or medicinal liquid agent with a known density (and/or surface tension) may still be retained in the fluid chamber 322 when exposed to normal (e.g., gravitational) forces.

According to some embodiments, the passage 324 may be configured as a plurality of conduits, holes, apertures, and/or other opening features. While a single passage 324 having the diameter “D” is depicted in FIG. 3A and FIG. 3B, for example, the diameter “D” may instead represent a cumulative opening diameter for a plurality of smaller opening features disposed at the second end of the fluid reservoir 322. In some embodiments, the passage 324 may also or alternatively be shaped to favor liquid flow in one direction versus the other and/or may be shaped to limit or prevent liquid flow in at least one direction. In such a manner, for example, the liquid filled in the BFS vial 310 may be advantageously and/or selectively retained in various portions or areas of the BFS vial 310, e.g., during the BFS manufacturing process, storage, transportation, and/or stages of use.

In some embodiments, fewer or more components 312, 312-1, 312-2, 312-3, 312-4, 312-5, 314, 316, 316-1, 316-2, 318, 322, 324 and/or various configurations of the depicted components 312, 312-1, 312-2, 312-3, 312-4, 312-5, 314, 316, 316-1, 316-2, 318, 322, 324 may be included in the portion of the BFS vial 310 without deviating from the scope of embodiments described herein. In some embodiments, the components 312, 312-1, 312-2, 312-3, 312-4, 312-5, 314, 316, 316-1, 316-2, 318, 322, 324 may be similar in configuration and/or functionality to similarly named and/or numbered components as described herein. According to some embodiments, the portion of the BFS vial 310 may comprise the mounting flange 316 but not the constriction 324. In some embodiments, the portion of the BFS vial 310 may comprise the constriction 324 but not the mounting flange 316.

### IV. BFS Mating Collars

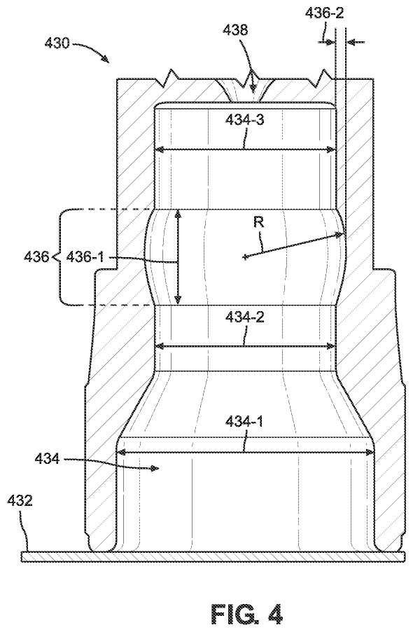

Turning to FIG. 4, a side cross-sectional view of a portion of an administration assembly 430 according to some embodiments is shown. In some embodiments, the administration assembly 430 may comprise a seal 432 disposed and/or coupled to seal an interior volume 434 at a first end thereof. In some embodiments, the interior volume 434 may be shaped to accept and/or retain a neck of a BFS vial (not shown in FIG. 4; e.g., the neck 112, 212, 312 of FIG. 1A, FIG. 1B, FIG. 1C, FIG. 1D, FIG. 2A, FIG. 2B, FIG. 2C, FIG. 2D, FIG. 3A, and/or FIG. 3B herein), e.g., by defining various internal dimensions and/or features. The interior volume 434 may comprise and/or define, for example, a first interior diameter 434-1, a second interior diameter 434-2, and/or a third interior diameter 434-3, each defined over different portions of the interior volume 434. According to some embodiments, the interior volume 434 (and/or the administration assembly 430) may comprise and/or define an interior mating feature 436 that is specially located, sized, and/or shaped to accept a mounting flange (not shown in FIG. 4; e.g., the “doughnut” shaped mounting flange 116, 216, 316 of FIG. 1A, FIG. 1B, FIG. 1C, FIG. 1D, FIG. 2A, FIG. 2B, FIG. 2C, FIG. 2D, FIG. 3A, and/or FIG. 3B herein). In some embodiments, the interior mating feature 436 may comprise an interior groove, channel, and/or seat that comprises and/or defines a mating length 436-1, a seat depth 436-2, and/or a rounding radius “R”. According to some embodiments, the interior volume 434 may comprise an effluent passage or exit 438 disposed, formed, and/or cut into a second end thereof.

According to some embodiments, the administration assembly 430 may be axially engaged to couple with a BFS vial (not shown) via application of an axial mating force. The seal 432 may be removed, for example, and the administration assembly 430 may be urged onto the neck of the BFS vial such that the cooperatively shaped interior mating feature 436 accepts the mounting flange of the BFS vial, thereby selectively and/or removably coupling the BFS vial to the administration assembly 430. In some embodiments (not shown), the interior mating feature 436 (and/or other interior features) and/or the mounting flange may be shaped such that uncoupling of the BFS vial and the administration assembly 430 is mechanically prohibited. According to some embodiments, the neck of the BFS vial may be advanced into the interior volume 434 at a first portion thereof having the first interior diameter 434-1. In some embodiments, the neck may continue to advance into a second portion of the interior volume 434 at a second portion thereof having the second interior diameter 434-2. As depicted, the second interior diameter 434-2 may be smaller than the first interior diameter 434-1. According to some embodiments, the second interior diameter 434-2 may be sized to accept an outer diameter of the BFS vial neck, which may continue to be inserted into the interior volume 434.

In some embodiments, once the mounting flange (e.g., exterior rounded and/or axially elongated flange) of the BFS vial reaches the second portion having the second interior diameter 434-2, the mounting flange will engage with the side walls at the juncture/transition between the first interior diameter 434-1 and the second interior diameter 434-2. The first interior diameter 434-1 may be sized, for example, to be larger than the radial extents of the mounting flange, but the second interior diameter 434-2 may be sized smaller than the radial extents, causing an engagement thereof. In some embodiments, such as depicted (but not separately labeled) in FIG. 4, an interior taper may be provided between the first interior diameter 434-1 and the second interior diameter 434-2, such that the mounting flange may engage along the taper, before the second portion with the second interior diameter 434-2 is reached.

According to some embodiments, the BFS vial may be softer than (e.g., have a lower hardness rating and/or be elastic) the material of the administration assembly 430, which may cause the mounting flange to deflect radially inward upon engagement (and continued application of axial force) with the inside walls/surfaces of the interior volume 434. The mounting flange may deform, compress, and/or flatten to pass through the second portion having the second interior diameter 434-2, for example, and may advance into the interior mating feature 436.

According to some embodiments, once the mounting flange of the BFS vial passes into the interior mating feature 436, the mounting flange may expand radially outward to (or near) the original axial extents thereof (e.g., releasing the elastic potential energy stored by the elastic deformation thereof). In some embodiments, such as in the case that the interior mating feature 436 is sized to be slightly smaller (e.g., one half to two percent (0.5%-2.0%)) than the mounting flange, the mounting flange may be able to reform only to near its original extents, thereby causing the mounting flange to retain some stored elastic energy due to continued (although small) deformation thereof. Such retained deformation may, for example, cause an interference pressure to remain between the mounting flange and the inside walls of the interior mating feature 436 such that the fit between the materials remains tight and substantially leakproof. In some embodiments, the configuration of the interior mating feature 436 and/or of the interior volume 434 may be defined to be cooperative with a specifically sized BFS vial or bottle (e.g., the BFS vial 310 of FIG. 3A and/or FIG. 3B herein) such that the mating thereof may be accomplished via a steady, uniform application of a mating force that both reduces strain on the components as well as provides for a successful and repeatable user experience.

According to some embodiments, the first interior diameter 434-1 may be sized between seven and forty-five hundredths millimeters and nine millimeters (7.45-mm to 9-mm). In some embodiments, the second interior diameter 434-2 may be sized between six and a half millimeters and seven millimeters (6.5-mm to 7-mm). According to some embodiments, the third interior diameter 434-3 may be sized equal to the second interior diameter 434-2 or may be sized smaller. The third interior diameter 434-3 may be sized, for example, between six millimeters and six and a half millimeters (6-mm to 6.5-mm). In some embodiments, the mating length 436-1, seat depth 436-2, and/or rounding radius “R” may be sized cooperatively with the mounting flange of the BFS vial. According to some embodiments, the mating length 436-1 may be sized between three and four millimeters (3-mm to 4-mm), the seat depth 436-2 may be sized between sixty-five hundredths and nine tenths millimeters (0.65-mm to 0.8-mm), and/or the rounding radius “R” may be between four and five millimeters (4-mm to 5-mm).

In some embodiments, fewer or more components 432, 434, 434-1, 434-2, 434-3, 436, 436-1, 436-2, 438 and/or various configurations of the depicted components 432, 434, 434-1, 434-2, 434-3, 436, 436-1, 436-2, 438 may be included in the administration assembly 430 without deviating from the scope of embodiments described herein. In some embodiments, the components 432, 434, 434-1, 434-2, 434-3, 436, 436-1, 436-2, 438 may be similar in configuration and/or functionality to similarly named and/or numbered components as described herein.

### V. Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention nor a listing of features of the invention that must be present in all embodiments.

Neither the Title (set forth at the beginning of the first page of this patent application) nor the Abstract (set forth at the end of this patent application) is to be taken as limiting in any way as the scope of the disclosed invention(s).

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “a”, “an” and “the” mean “one or more”, unless expressly specified otherwise.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality/features.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Headings of sections provided in this patent application and the title of this patent application are for convenience only, and are not to be taken as limiting the disclosure in any way.

“Determining” something can be performed in a variety of manners and therefore the term “determining” (and like terms) includes calculating, computing, deriving, looking up (e.g., in a table, database or data structure), ascertaining and the like

The terms “including”, “comprising” and variations thereof mean “including but not limited to”, unless expressly specified otherwise. As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as “inner,” “outer,”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A pre-filled medical delivery device, comprising:
a blow-fill-seal (BFS) vial defining a collapsible fluid chamber, a dispensing chamber in communication with the collapsible fluid chamber, a neck portion in communication with the dispensing chamber, and an exterior flange formed on the neck portion, wherein the exterior flange comprises an axially elongated and rounded protrusion having a flange length in the range of thirty to forty-five percent (30%-45%) of a length of the neck portion; and
an administration assembly defining an opening at one end, the opening being in communication with an interior volume comprising an interior seat into which the exterior flange is axially mated, and the administration member comprising, at a second end, a needle.

2. The pre-filled medical delivery device of claim 1, wherein the BFS vial further comprises:
a constriction between the fluid chamber and the dispensing chamber.

3. The pre-filled medical delivery device of claim 2, wherein the constriction comprises a diameter in the range of three to four millimeters (3-mm to 4-mm).

4. The pre-filled medical delivery device of claim 1, wherein the exterior flange is rounded at a radius in the range of four to five millimeters (4-mm to 5-mm).

5. The pre-filled medical delivery device of claim 1, wherein the exterior flange extends radially outward from the neck portion by a protrusion amount and wherein the flange length is in the range of four and three tenths times (4.3×) and five and three tenths times (5.3×) the protrusion amount.

6. A blow-fill-seal (BFS) vial, comprising:
a collapsible fluid chamber;
a dispensing chamber in communication with the collapsible fluid chamber;
a cylindrical neck portion in communication with the dispensing chamber; and
an exterior flange formed on the neck portion, wherein the exterior flange extends radially outward from the neck portion by a protrusion amount and wherein an axial length of the exterior flange is in the range of four and three tenths times (4.3×) and five and three tenths times (5.3×) the protrusion amount.

7. The BFS vial of claim 6, wherein the axial length of the exterior flange is in the range of thirty to forty-five percent (30%-45%) of a length of the neck portion.

8. The BFS vial of claim 6, wherein the BFS vial further comprises:
a constriction between the fluid chamber and the dispensing chamber.

9. The BFS vial of claim 8, wherein the constriction comprises a diameter in the range of three to four millimeters (3-mm to 4-mm).

10. The BFS vial of claim 6, wherein the exterior flange is rounded at a radius in the range of four to five millimeters (4-mm to 5-mm).

11. The BFS vial of claim 6, wherein at least one of the collapsible fluid chamber, the dispensing chamber, and the cylindrical neck is filled, during manufacture, with a volume of liquid.

12. The BFS vial of claim 11, wherein at least one of the collapsible fluid chamber, the dispensing chamber, and the cylindrical neck also comprises a volume of gas.

13. The BFS vial of claim 12, wherein the volume of gas is in the range of one hundred to two hundred and fifty percent (100%-250%) of the volume of liquid.
